(19) United States
(12) Patent Application Publication
(10) Pub. No.: US 2015/0246082 A1
(43) Pub. Date: Sep. 3, 2015

(54) Pediococcus-Based Probiotics for Body Weight Control

(71) Applicant: IMAGILIN TECHNOLOGY, LLC, Frederick, MD (US)

(72) Inventor: Jhy-Jhu LIN, Potomac, MD (US)

(21) Appl. No.: 14/714,042

(22) Filed: May 15, 2015

Related U.S. Application Data
(63) Continuation-in-part of application No. 13/736,455, filed on Jan. 8, 2013.
(60) Provisional application No. 61/996,710, filed on May 15, 2014.

(51) Int. Cl. A61K 35/744; A23G 1/56; A23C 9/152; A23L 1/30; A23L 1/212

(52) U.S. Cl. CPC A61K 35/744; A23L 1/3014; A23L 1/2128; A23C 9/152; A23G 1/56; A23L 1/2121; A23V 2002/00; A23Y 2280/15

(57) ABSTRACT

A method of augmenting a weight-loss regimen composition comprising administering at least one billion P. acidilactici NRRL B-50517 daily in a carrier.

### CROSS-REFERENCE TO RELATED APPLICATIONS

This application is a continuation-in-part of U.S. application Ser. No. 13/736,455, filed Jan. 8, 2013 in the U.S. Patent and Trademark Office and takes priority from U.S. Provisional Patent Application No. 61/996,710, filed May 15, 2014. All disclosures of the document(s) named above are incorporated herein by reference.

### BACKGROUND AND FIELD OF THE INVENTION

This invention relates to the field of probiotics and, with particularity, to P. acidilactici and compositions containing P. acidilactici and compositions containing said organisms. The use of probiotics to provide restorative health to the intestine by restoring useful bacteria and, sometimes, replacing undesirable bacteria, has been known. The P. acidilactici containing compositions of this invention are particularly useful in treatment of various intestinal conditions and in improving weight loss. The invention disclosed herein provides compositions containing P. acidilactici strains that are particularly useful, since these probiotic strains are resistant to destruction from excess salt, sugar and acids and are also more resistant to destruction than other strains of probiotic organisms at temperatures used for pasteurization. The organisms also persevere in dry environments. Hence, there is more possibility for making a wide variety of compositions for administration to mammals (including humans) and birds.

Live microorganisms have been given as nutritional supplements to animals and humans since historical times. For example, yeast has long been used as a nutrient supplement for livestock and humans. Yogurt, which contains desirable bacteria (Lactobacillus and/or Bifidobacterium) is commonly used to prevent and cure diarrhea-related gastrointestinal diseases. Many probiotics have also been used to combat infections arising from undesirable intestinal flora, to enhance beneficial immune responses and to enhance general intestinal health, including intestinal mobility.

While Lactobacillus, which is naturally present in yogurt, has long been known and taught as beneficial for intestinal health and well-being, there are many instances when its use is problematic. Yogurt does not have a particularly long shelf life and, as a milk product, is not useful for all humans or other species of animals that are milk intolerant. The compositions of the invention present many advantages. Using methods of the invention, probiotic-containing products can be pasteurized for long shelf life. Additionally, the addition of sugars and salt to enhance taste may be used in the probiotic compositions of the invention to an extent that was not possible using previous strains and methods of preparation. The compositions of the invention are particularly useful, since they are more acceptable by reason of palatability. Of particular value is the Pediococcus acidilactici (deposited in the Agricultural Research Service (ARS) Patent Culture Collection as NRRL B-5051)

The process of encapsulation of probiotics was described in U.S. Pat. No. 8,496,923 of Lin, which is incorporated herein by reference in its entirety. The compositions of that patent involved encapsulation of the P. acidilactici and Saccharomyces baulardii to make compositions for administration to animals and birds. The encapsulated organisms were administered for purposes of treating infectious diseases of the gastro-intestinal tract and for enhancing weight loss.

Published Patent Application 20130064885 of Lin discloses P. acidilactici encapsulated in a gelatin capsule. The compositions of the present invention are not disclosed therein.

U.S. Pat. No. 8,793,895 to Gasteyer, et al., which is incorporated by reference herein in its entirety, teaches a lyophilization system and method which may be used to produce the products of the invention.

U.S. Pat. No. 7,888,062, which is incorporated by reference herein in its entirety, teaches a method of lyophilization of Lactobacillus acidophilus and compositions containing the lyophilized organisms. The lyophilization methods taught therein are appropriate for use in lyophilization of the P. acidilactici for use in the compositions of this invention.

The above are merely some of the disclosures that provide techniques for lyophilization for use in making the compositions of the invention. However, the methods taught therein do not provide the compositions of the invention, but merely teach methods commonly used in lyophilization that are appropriate for practicing the invention.

The above does not exhaustively recite the literature containing teaching of use of probiotics. However, that literature gives background useful in practice of the invention. The practice of the invention uses optimized strain of Pediococcus acidilactici (deposited in the Agricultural Research Service (ARS) Patent Culture Collection as NRRL B-5051) that is viable above 65° C. and can, in many cases, be subjected to temperatures of 85° C. without being destroyed. The strain is able to grow in a pH range between 1 and 6.2, and is also able to grow in aerobic and anaerobic conditions. This will increase both the efficiency of the methods for manufacturing probiotics and the integration of probiotics into food/feed manufacturing processes, which often require high temperatures.

### SUMMARY OF THE INVENTION

It is the purpose of this invention to provide compositions containing probiotic organisms in compositions that retain at least 10⁹ P. acidilactici organisms per dose of the product. Several carriers, including fruit sauces, are provided. (More commonly the compositions will contain at least 5×10⁹ organismsper dose.) The compositions containing the active organisms can be, for example, milk products, fruit juices or vegetable juices. Once a composition of any ingestible product containing the appropriate amount of P. acidilactici has been produced, the ingestible product may be added to various carriers for making products. Such carriers include fruit juices, pureed fruits and vegetables, milk products (flavored or unflavored) puddings, cereals, formulas particularly for use in feeding the elderly and children, confections and carbonated drinks. An interesting use is in compositions of colostrum or compositions used as replacement for colostrum in very young mammals. (Such products containing P. acidilactici can be used in treating orphaned mammals.)

### BRIEF DESCRIPTION OF THE DRAWINGS

FIG. 1 shows the effects of use of the product of Example 1 on the participants in a health study.

FIG. 2 shows the effects of the product of Example 1 on body weight of participants with regular diets.

FIG. 3 shows the effects of the product of Example 1 on weight of participants under the hCG diet program.

### DETAILED DESCRIPTION OF THE INVENTION

### Materials and Methods: Preparation and Evaluation of Samples

### Preparation of MRS Agar Medium

MRS agar broth and plates were prepared by adding the individual components of the medium to required concentrations, which was made up to 1 liter with de-ionized water The pH was then adjusted as necessary to 6.2 using sodium hydroxide or hydrochloric acid as required (Media composition 2008)

### Dilution and Spread Plate Technique

Dilution and plating of samples is a method for determining the number of bacteria present in a particular sample and most importantly to isolate individual colonies. For this study, 0.5 g powder packets of Mitogrow, a commercially available form of the Pediococcus acidilactici strain IMPA was taken as a sample and dissolved in 5 ml of 1× Phosphate Buffered Saline (PBS) solution. The solution was then mixed thoroughly by vortex stirring at regular intervals for up to 15 minutes. From this sample, 10 μl of sample was taken and diluted in 990 μl of PBS solution in an Eppendorf tube. This is the 10⁻² dilution of the sample. From the 10⁻² dilution, 10 μl of sample was taken and diluted again in 990 μl of PBS solution, which gives the 10⁻⁴ dilution. This procedure was repeated taking 10 μl from the 10⁻⁴ dilution as a diluent, thus obtaining the 10⁻⁶ dilution From the 10⁻⁶ dilution, 100 μl of sample was taken and diluted in 900 μl of PBS solution to give a dilution of 10⁻⁷. And, finally from the dilution tube, another 100 μl of sample was taken and diluted in 900 μl of PBS solution, to obtain a dilution of 10⁻⁸.

A PBS tube without any inoculation of the sample was used as a control. By the above method several dilutions of a sample were obtained, which were then cultured onto a pH 6.2 MRS agar plate by adding 100 μl from the 10⁻⁶, 10⁻⁷ and 10⁻⁸ tubes and spreading them using glass beads. The plates were incubated at 45° C. for 24 hours, after which the number of colonies can be determined by a direct and viable counting method known as Heterotrophic plate count (Cell Enumeration 2009). In this method, the number of colonies grown were counted and used to estimate the number of bacteria in the initial sample by the following formula:

B=N/D where,

B=number of bacteria

N=number of colonies counted on a plate

D=dilution factor (1, 10 or 100).

High-Temperature Resistant Selection of P. acidilactici

Following the above technique, individual colonies were isolated. A single colony was taken using a sterile toothpick and introduced into 5 ml of MRS agar broth in 15 mL falcon tubes. Several individual colonies were introduced into separate tubes in a similar fashion, so as to increase the amount of the starter culture and the chances of isolating a temperature-resistant strain. These tubes were incubated at 65° C. in a Hot Water Bath incubator for 24-48 hours. An incubation time of 24-48 hours was employed in order to establish that there had been survival among the Pediococcus acidilactici that came from a single colony. It is also a way of enriching the surviving bacteria in such a way that the ensuing progeny will be more resistant to heat than the previous generation, enhancing the chances of obtaining a strain that is not only resistant to 65° C. but also to even higher temperatures.

To determine whether there had been any growth of P. acidilactici in the tubes incubated at 65° C. for 24-48 hours, 100 μl of broth from each tube was cultured onto MRS agar plates by Spread Plate Technique and the number of colonies estimated after incubation at 45° C. overnight. It is also ideal to determine the number of colonies in the initial single colony taken for selection so to estimate the survival rate of the bacteria during the course of the heat treatment and after its completion This was done by culturing 100 μl of broth from the tubes onto MRS agar plates and incubating them at 45° C. for 24 hours, immediately after inoculation into 5 ml of MRS agar broth and also at regular time intervals prior to and during the 24-48 hour period of heat treatment to observe the change in number of cells over time.

If there is any growth, isolated colonies from the MRS agar plates were then introduced again into 5 ml MRS broth tubes and incubated again at 65° C. so as to establish a replicate and also to verify the viability of the bacteria. The same procedure leads to the amplification of the bacteria which can survive at the pre-set temperature. Once the procedure was repeated and colonies were isolated again, the temperature was raised to 70° C. and the experiment was repeated. This process was repeated by increasing the selection temperature until a strain that survived at 85° C. was developed. The strains thus isolated were recorded as IMPA-I, IMPA-2 and so on.

### Low-pH Tolerance Selection:

The goal of the study was to isolate a strain which is capable of surviving both high temperatures and low pH levels. With this in mind the Pediococcus acidilactici strain obtained through temperature resistant selection was used as the starter culture for pH selection. Once selection for temperature resistance was achieved in the form of a desired strain which was capable of surviving at a very high temperature, such as 85° C., single colonies of Pediococcus acidilactici obtained through the dilution and spread plate technique was subjected to pH tolerance by inoculating the culture into MRS broths of different pH levels MRS ranging from 1.0 to 6.2.

Similar to the temperature resistance method above, the colonies obtained after initial culturing of the 85° C. strain were subjected to subsequent pH selection by inoculating them into a 2.5 pH MRS broth and incubating it at 45° C. for 24 hours. In the case of pH selection, the time of incubation was limited to 24 hours based on previous research results (Lin, 2006). Once the number of colonies was estimated the procedure was repeated again at the same pH level to observe if there was any growth of the microorganism. This helped in isolating viable cultures that are tolerant to 2.5 pH levels. A method of enriching the selected strain was to inoculate it in a MRS broth set at a comfortable pH level such as 4.5 and then re-introduce the cultured sample to the pH level of selection, i.e., once colonies survived and grew in 2.5 pH MRS broth, in order to enrich it, the sample was cultured for 24 hours MRS broth at 4.5 pH and then re-selected again at pH 2.5 This method was repeated at a pH level of 1.5 using MRS broth and was very effective in the amplification of Pediococcus acidilactici that tolerates pH 1.5.

### Preparation of Glycerol Stock:

Selected strains of Pediococcus acidilactici that survived at the different temperatures, especially IMHP that survived at both 85° C. and pH 1.5 were preserved for further studies. Therefore, it was necessary to establish a method in which to store the viable bacterial cells. One of the techniques widely used is the quick-freezing method, wherein the viable cells are stored at −70° C. after suspending them in a mixture of growth media and a stabilizing protectant such as glycerol. It has been observed that freezing bacteria directly without any protection can cause loss of viability during the freezing and subsequent thawing process due to mechanical and physiological conditions (Morrison 1979). Accordingly, in a preferred embodiment of the invention, glycerol was used as a protecting agent for the process of freezing.

In order to freeze and store the selected Pediococcus acidilactici cultures, first the samples are cultured using the spread plate technique in a 5 ml falcon tube consisting of MRS broth after incubation at 45° C. overnight. It is important that the sample be prepared for freezing after overnight incubation and not 24 hours, since the bacteria in the overnight incubation sample are still in the ‘log’ phase of the growth curve. This helps in establishing a sample that can readily grow to maximum potential after it is thawed. Subsequent to freezing 300 μl of the sample was in a labeled cryogenic tube, to which was added 300 μl of a mixture solution containing 50% MRS broth and 50% glycerol. The mixture solution containing glycerol and MRS broth in equal amounts was prepared using sterile glycerol and MRS broth before the experiment was set up. The cryogenic tube containing the cultured sample of Pediococcus acidilactici and the glycerol-MRS broth mixture was thoroughly mixed by vortex and immediately set into dry ice or into the −70° C. freezer to initiate the freezing and storage process. Several cryogenic tubes were set up in similar fashion and hence are termed, ‘primary seed’. These tubes can be used as and when required to cultivate the selected strain of Pediococcus acidilactici. During the process of preparing glycerol stock 100 μl of the initially cultured sample was diluted and plated onto MRS agar plates for incubation at 45° C. for 24 hours. The product was used to compare the survival of the cryogenically frozen samples which were also diluted and plated onto MRS agar plates and incubated at 45° C. for 24 hours. By this method it was possible to ascertain if the freezing process affected the survival of the selected strain of Pediococcus acidilactici (IMHP) capable of surviving at 85° C. and at a low pH of 1.5.

### Comparison of Aerobic and Anaerobic Growth Characteristics:

Production of probiotics involves certain techniques such as conventional batch fermentation procedures, continuous culture and immobilized cell system technologies (Heller 2001). The Pediococcus acidilactici which are facultative anaerobes that are capable of surviving in the presence and also in the absence of oxygen (Facklam and Elliot 1995). However, for production purposes, it is ideal to identify a strain that produces high yields of culture under both aerobic and anaerobic conditions. In order to determine the most optimal conditions of growth it is necessary to compare the difference in growth characteristics of various strains of Pediococcus acidilactici with the parent strain as well as obtaining the ideal strains selected for survival at high temperatures and low pH. The strain obtained after the resistance selection methods, which was capable of surviving 85° C. and pH 1.5 was cultured onto MRS agar plates and a single colony was introduced to 25 ml tubes containing MRS agar broth. Another sample of culture was taken from the parent type Pediococcus acidilactici, incubated onto MRS agar plates and inoculated into 25 ml MRS tubes as before. Two separate tubes were set up for aerobic and anaerobic testing. The aerobic tube was incubated for 24 hours in an environmental shaker at a temperature of 45° C. The shaking speed was set at 250 rpm, in order to induce aeration. Meanwhile, 1 ml of mineral oil was added on top of the broth in the anaerobic tubes and the mouths of the tubes were sealed with tape to avoid to oxygen or air exchanges. The tubes were incubated for 24 hours in the regular incubator at 45° C. in a still position in order to avoid aeration.

Growth characteristics of the regular Pediococcus acidilactici were also determined by the same procedure using the bacteria from Mitogrow samples, which acts both as a control and a means of comparison between the wild type strain and the strain resistant to temperatures over 85° C. and low pH. 0.5 g of Mitogrow powder (0.5 g) was dissolved into 5 ml of 1×PBS solution, serially diluted and then plated onto MRS agar plates and incubated at 45° C. for 24 hours. A single colony was then subjected to the same treatment of aerobic and anaerobic conditions as discussed above.

At incubation intervals of time, 1 ml of the broth was withdrawn from the tubes and the optical density was measured using a Spectrophotometer set up at 600 nm wavelength. The idea was to measure the amount of light that passes through a liquid culture, to estimate the number of cells/ml based on the amount of light that passes through. This is an indirect and total method to determine not only the growth of Pediococcus acidilactici but also can be used to observe any amplification during the procedure.

Physiological Comparison Parent Pediococcus acidilactici Strain and Selected Strain of Pediococcus acidilactici (IMHP):

Samples from IMPA and ATCC Pediococcus acidilactici strain 25743 purchased from American Type Culture Collection (ATCC), Manassas, Va., and the above strain selected for 85° C. and pH 1.5 survival were subjected to temperature tolerance selection and also to pH selection. 10 μl of the sample was suspended into 490 μl of MRS media broth in an Eppendorf tube with different pH and incubated at 85° C. up to 24 hours. The number of colonies within the initial inoculated sample was determined by plating 100 μl of broth immediately after inoculation onto MRS agar plates using spread plate technique and incubated at 45° C. for 24 hours. This gives a count of the number of colonies in the starting population before the treatment. After the 85° C. treatment 100 μl from each of the Eppendorf tubes was plated on to MRS agar plates. The total number of colonies was counted from the plates after incubating them for 24 hours at 45° C., which gives the number of colonies; an indication of the similarity or difference between the survivability and replicating capabilities of the strains.

Simultaneously, the ATCC 25743 strain, IMPA and the IMHP Pediococcus acidilactici strains were inoculated into 1 ml of MRS broth taken in Eppendorf tubes and compared to response when subjected to an environment with 1.5 pH for the pH tolerance selection. This provided a confirmatory method and, by using the spread plate technique, a quantitative measure of the differences between the wild type and the temperature and pH resistant strains of Pediococcus acidilactici was possible. This study was replicated three times to establish a set of confirmatory data regarding the comparisons between the number of colonies in the starting population and the number of colonies after the desired treatment of both the wild type and the selected strains of Pediococcus

Pilot IMHP Pediococcus acidilactici Strain Scale-Up Experiment at 85° C.

Into a set of two 250 μl flasks, 50 μl of MRS broth were prepared and sterilized prior to the inoculation. Into each of these flasks 100 μl of IMHP primary seed stored at −70° C. was added as inoculum. The flasks were then incubated at 45° C. for 24 hours to allow maximum growth of the culture to produce a large batch of Pediococcus acidilactici. From this large batch of sample, 10 ml of sample was extracted and introduced into two separate 15 ml falcon tubes. The tubes containing 10 ml of the sample were then centrifuged for 15 minutes at an rpm of 2500.

After centrifugation a pellet was formed at the bottom of the tube while the supernatant remains at the top. The supernatant is discarded and the process is repeated so as to concentrate the pellet. The pellet from each of the tubes was then re-suspended into 2 ml MRS broth. Then 100 μl of the sample was plated onto MRS agar plates and incubated for 24 hours at 45° C. to provide a measure of the initial inoculum and determine the survival rate of Pediococcus acidilactici that is subjected to the temperature selection. The tube with the pellet was thoroughly mixed and incubated at 85° C. for 24 hours. After the incubation period the samples were cultured through the dilution and spread plate technique at up to 10⁻⁴ dilution and plated onto MRS plates which were incubated at 45° C. for 24 hours.

Unique Properties of P. acidilactici NRRL B50517:
Resistance to high osmotic environment.

### Example 1

### Survival of P. acidilactici NRRL B-50517 at Different Concentration of Lactose Solution

Number of P. acidilactici viable cells*

| % lactose | Day 0 | Day 1 | Day 3 | Day 9 |
|---|---|---|---|---|
| 10% | 1.2 × 10⁸ | 2.1 × 10⁸ | 1.9 × 10⁸ | 1.5 × 10⁸ |
| 20% | 1.8 × 10⁸ | 1.4 × 10⁸ | 2.4 × 10⁸ | 2.0 × 10⁸ |
| 30% | 2.3 × 10⁸ | 1.6 × 10⁸ | 4.3 × 10⁸ | 1.5 × 10⁸ |
| 40% | 4.9 × 10⁸ | 3.7 × 10⁸ | 3.3 × 10⁸ | 3.8 × 10⁸ |
| 50% | 1.8 × 10⁸ | 1.2 × 10⁸ | 1.0 × 10⁸ | 1.5 × 10⁸ |

### Example 2

### Survival of P. acidilactici NRRL B-50517 and E. coli at Highly Osmotic Solution

Numbers of P. acidilactici viable cells*

| | Incubation time | | | | | |
|---|---|---|---|---|---|---|
| | 24 hours | | 48 hour | | 72 hours | |
| Solution | E. coli | P. acidilactici | E. coli | P. acidilactici | E. coli | P. acidilactici |
| 20% lactose | 0 | 2.80 × 10⁶ | 0 | 1.40 × 10⁶ | 0 | 1.51 × 10⁶ |
| 50% sucrose | 0 | 3.15 × 10⁶ | 0 | 4.0 × 10⁶ | 0 | 2.75 × 10⁶ |
| 20% NaCl | 0 | 2.75 × 10⁶ | 0 | 2.65 × 10⁶ | 0 | 2.70 × 10⁶ |
| 10% lactose and | 0 | 2.75 × 10⁶ | 0 | 3.05 × 10⁶ | 0 | 3.00 × 10⁶ |
| 10% NaCl | | | | | | |
| Saline (0.1% NaCl) | 1.2 × 10⁵ | 4.15 × 10⁶ | 9.05 × 10⁴ | 2.40 × 10⁶ | 4.0 × 10⁴ | 1.5 × 10⁵ |

*five colonies were isolated from the overnight culture of E. coli DH5α, and P. acidilactici NRRL B-50517 into 1 ml of saline in a 1.5 ml microcentrifuge tube, vortexed to mix well solution, and transferred 100 ul bacterial solution into a falcon tube with 5 ml of different high osmotic solution, placed the tubes at room temperature for 24 hours, 48 hours and 72 hours. After incubation, performed series of dilution as required, and plated 100 ul of desirable dilution onto LB plates for E. coli, and MRS plate for P. acidilactici. Counted the numbers of bacteria after overnight incubation.

Survival of P. acidilactici NRRL B-50517 after the Dispersion of Different Concentration of 85C Sucrose Solution into the Containers with P. acidilactici NRRL B-50517 Dried Powders.

| | # of P. acidilactici after exposure |
|---|---|
| % sucrose heated at 85° C. | to 85° C. sucrose solution* |
| 10% sucrose | 7.62 × 10⁶ |
| 20% sucrose | 2.18 × 10⁶ |
| 30% sucrose | 1.24 × 10⁷ |
| 40% sucrose | 1.29 × 10⁷ |
| 50% sucrose | 1.97 × 10⁷ |

*Sucrose, 5 ml of solution was placed into a 15 ml falcon tube and incubated at 87 C. in a water bath. The temperature of the sucrose solution in a falcon tube was determined by inserting a digital thermometer directly into the sucrose solution. After the sucrose solution reached at 85° C. or higher, the sucrose solution was transferred to a 15 ml falcon tube with 0.2 g P. acidilactici NRRL B-50517 powders (1 billion cfu/g) at room temperature. The falcon tube was vortexed to mix powders with sucrose solution well, and placed the tube at room temperature for 20 min. A 2 ml sample of sucrose solution with P. acidilactici was transferred to a new a 15 ml falcon tube with 4 ml saline, the falcon tube was vortexed to mixed well, and performed a series of dilutions in saline were prepared. The 100 ul diluted samples were plated onto MRS agar plates. The colonies were counted after the overnight incubation.

Survival of P. acidilactici NRRL B-50517 at Different Concentration of Lactose Solution:

Lactose in amounts of 2 g, 4 g, 6 g, 8 g, and 10 g of lactose were dissolved in up to 20 ml water to make 10%, 20%, 30%, 40% and 50% lactose solutions. The lactose solutions were sterilized by standard methods at 121° C., 15 Psi for 15 min.

The solutions were then transferred in 5 ml amounts of 10%, 20%, 30%, 40% and 50% sterilized lactose solution into a 15 ml falcon tube with 0.2 g P. acidilactici NRRL B-50517 dried powders separately and vortexed to make sure that the bacterial powders completely dissolved into lactose solution. The tubes with the bacteria in lactose solution were kept at room temperature. Standard bacterial viability assays were performed by series of dilutions with saline at day 0, day 1, day 3, and day 9. Samples with 100 ul diluted bacterial solution were plated onto MRS plates for P. acidilactici. The numbers of bacteria were counted after overnight incubation.
Survival of P. acidilactici NRRL B-50517 and E. coli at Highly Osmotic Solution:

Five colonies were isolated from the overnight culture of E. coli DH5α, and P. acidilactici NRRL B-50517 and injected into 1 ml of saline in a 1.5 ml microcentrifuge tube. The tubes were vortexed to mix the solution well, and the contents were transferred into 100 ul bacterial solution in a falcon tube with 5 ml of different high osmotic solution. The tubes were kept at room temperature for 24 hours, 48 hours and 72 hours. After incubation, a series of dilution were prepared as required, and 100 ul of desirable dilution was applied onto LB plates for E. coli, and MRS plate for P. acidilactici. After being incubated over night the bacteria were counted.

Survival of P. acidilactici NRRL B-50517 Disbursed into Different Concentrations of 85C Sucrose Solution into the Containers with P. acidilactici NRRL B-50517 Dried Powders.

Five ml sucrose solution was placed in a 15 ml falcon tube and incubated at 87° C. water bath. The temperature of the sucrose solution in a falcon tube was determined by inserting a digital thermometer directly into the sucrose solution. After the sucrose solution reached at 85° C. or higher, the sucrose solution was transferred to a 15 ml falcon tube with 0.2 g P. acidilactici NRRL B-50517 powders (1 billion cfu/g) at room temperature. The falcon tube was vortexed to mix the powder with sucrose solution well and the tube kept at room temperature for 20 min. Thereafter, 2 ml of the sucrose solution with P. acidilactici was transferred to a new a 15 ml falcon tube with 4 ml saline, vortexed and mixed well. Then a series of dilutions of saline were made. Then 100 ul diluted samples were plated onto MRS agar plates. The colonies were counted after the overnight incubation. Results recorded were based on the average of three times repeated experiments.

### Example 1

Pouches of apple sauce were preheated to 50° C. for 30 minutes and at 85° C. or 90° C. for 30 minutes. (The temperature of the apple sauce was checked with a digital thermometer to make sure the temperature of apple sauce was at 85° C. or 90° C.) Two 50 ml Falcon tubes were prepared with 1 g of P. acidilactici NRRL B-50517. The freeze dried powder containing at least 1 billion CFU/g of organisms was added to 20 ml of 85° C. or 90° C. apple sauce. The product was then vortexed for 20 minutes to mix the apple sauce with P. acidilactici dried powders. The product was then tested for viability. In the composition containing at least 1 billion CFU/g of the organisms the apple sauce acted as a carrier for the organisms to provide a readily administered product.

### Example 2

Pouches of milk were pasteurized. The milk was, after pasteurization, heated again and the process of inoculation with P. acidilactici was performed in the method of Example 1 was repeated except that the apple sauce was replaced with heated, pasteurized milk as a carrier.

### Example 3

A composition is prepared as in Example 1 wherein the carrier is a colostrum substitute.

### Example 4

A composition is prepared as in Example 1 except that the apple sauce is replaced with a nutrition supplement for administration to undernourished or elderly persons

### Example 5

A composition is prepared in accord with example 1 wherein the carrier, instead of an apple sauce, is a warm jellied product such as a gelatin.

It is also possible to prepare the P. acidilactici NRRL B-50517 in powder form to be added to powders of various flavors. The products may then be administered in capsular form or dissolved in a liquid such as water or seltzer water.

### Example 6

A powder containing P. acidilactici NRRL B-50517 was obtained by freeze drying the fermentation culture containing dextrose and P. acidilactici NRRL B-50517. The powder was used in preparation of the composition of Example 7.

### Example 7

Capsules containing at least 2 billion organisms of P. acidilactici NRRL B-50517 and peach fruit powder, which serves as a carrier, were prepared. A blend containing 100 g of B-50517 freeze dried powders (100 Billion CFU/g) and 2 kg of sprayed dried peach powders was mixed to produce a homogeneous mixture. The mixture was then encapsulated, each capsule containing 0.5 g of the mixtures of 50517 powders and peach powders in a gelatin capsule. This makes a capsule containing 2 billion CFU P. acidilactici B-50517 per capsule.

### Clinical Evaluation:

The compositions of Example 6 were tested in volunteers. The volunteer program was established by recruiting participants who visited either Sylvania Institute in Frederick, Md. or Natural Health and Acupuncture Clinic in Frederick, Md. Excluded were persons under 18 years of age, women who were pregnant or nursing, persons who are involved in current substance abuse, persons with a psychiatric disorder or other conditions which, in the opinion of the investigators, would impede compliance or hinder completion of the study. There were 12 participants on a regular diet and 15 on the hCG diet program. (See www.hcqmealplan.com) All participants were given daily doses of the product of example 1. The participants were asked to complete questionnaire forms relating to measured body weight, appetite, energy level, bowel movements, stool quality, vomiting, bloating and gas when they visited the clinics. In addition, the medical staff would perform weekly follow-up conversations with the participants by phone to collect data if the participants could not visit the clinics on schedule.

### Studies:

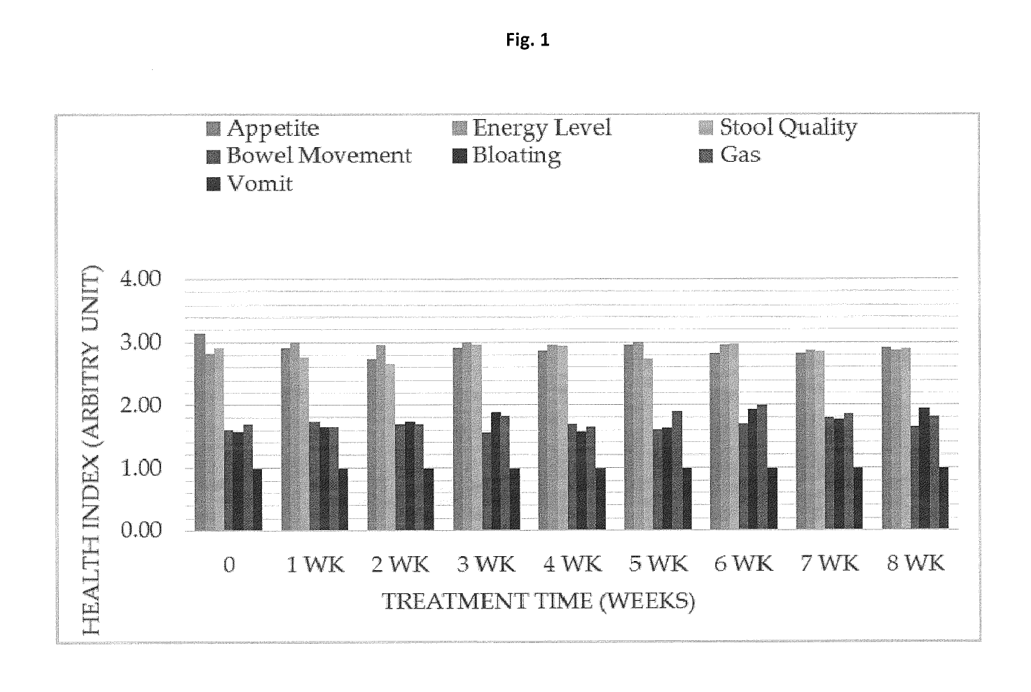

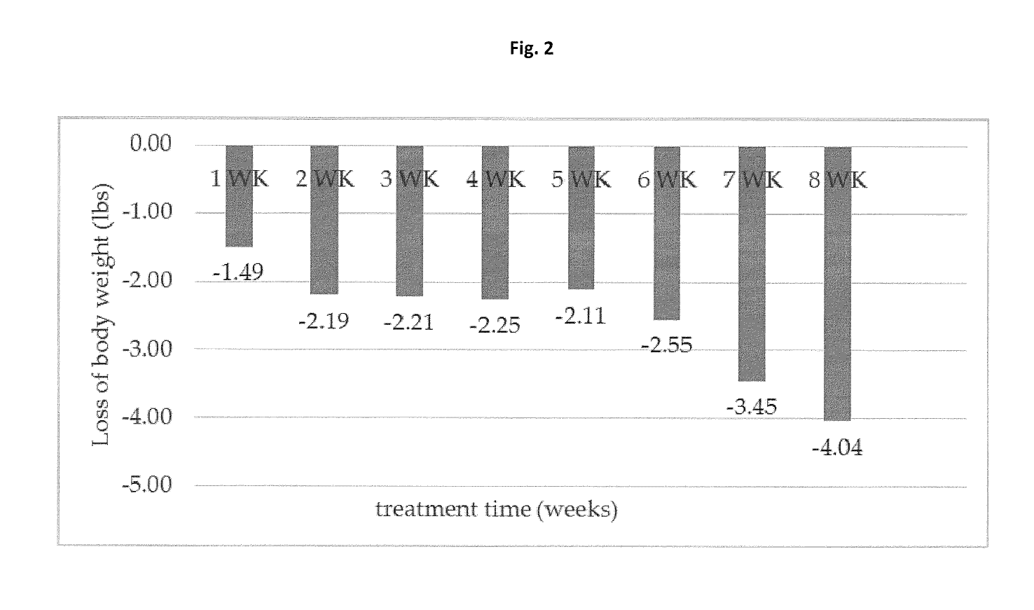

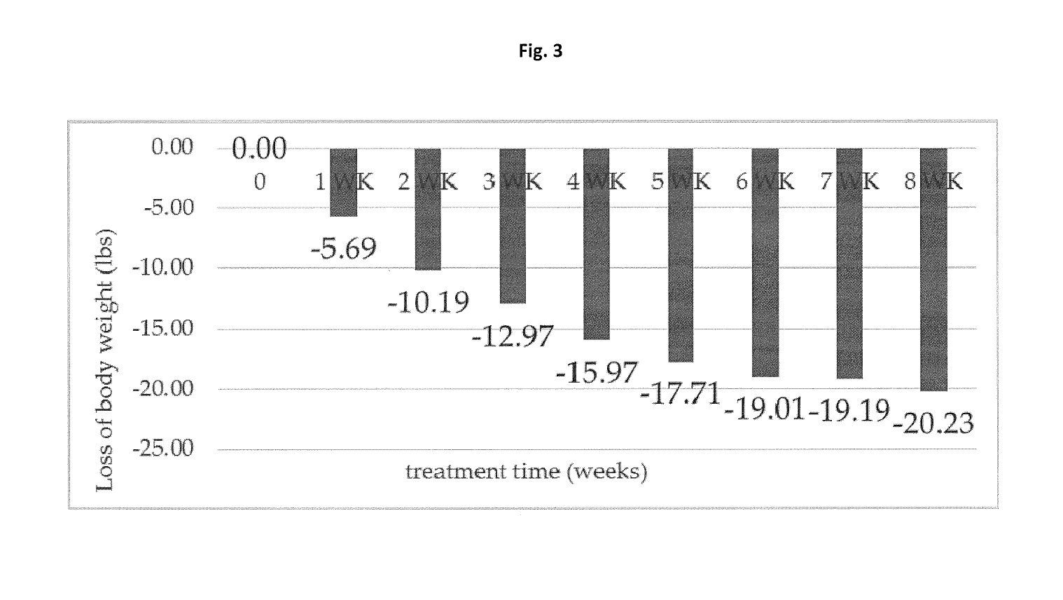

FIGS. 1, 2 and 3 show results of administration of a compound of the invention to persons who were seeking weight loss. The P. acidilactici NRRL B-50517 fermentative cultures were administered with peach fruit powders in gelatin capsules. The participants were required to complete questionnaire forms that I included measurement of body weight, appetite, energy level, bowl movement, stool quality, vomiting, bloating and gas. During the 8 weeks covered by the study there was no increase in any of the symptoms studied in those receiving the products of the invention.

When evaluating weight loss, it was found that those receiving the compositions of the invention (the product of Example 7) containing the P. acidilactici organisms while on either a regular or extreme diet (The hCG diet) lost weight more quickly than those who did not While taking the composition of Example 7 there were no significant differences in appetite, energy level, bowel movement, bloating or gas between those taking the product of Example 5 and those who received no product containing P. acidilactici NRRL B-50517 during the 8 weeks of the study. Interestingly, it was observed that weight loss while on the regular diets was about 4 pounds during the eight week period. The weight loss for participants on the hCG diet was, for those taking the encapsulated P. acidilactici NRRL B-50517 over the 8 week period, about 20 pounds. Interestingly, from these studies it appeared that participants suffering from overweight or obesity were benefited more than those who were of more nearly normal weight.

While the use of the encapsulated products containing P. acidilactici NRRL B-50517 is certainly convenient for certain purposes, it is also possible to encapsulate the freeze-dried P. acidilactici NRRL B-50517, then, when desired, empty the contents of the capsule into any carrier. For example, the freeze dried product may be delivered in pureed, liquid or soft, homogeneous products such as puddings being used as carriers.

An additional value is when the organisms are in sucrose solution they may be added to sauces, milk products or other liquid, semiliquid or pureed materials and heated, as exemplified above, to temperatures required for pasteurization. In this manner, large quantities of products such as milk, chocolate milk, fruit juices, etc. can be prepared and packaged for distribution and sale as products whose value is enhanced by the presence of the P. acidilactici NRRL B-50517 to improve digestion.

What we claim is:

1. A method of augmenting a weight-loss regimen composition comprising administering at least one billion P. acidilactici NRRL B-50517 daily in a carrier.

2. The method of claim 1 wherein the carrier is a pureed fruit or vegetable.

3. The method of claim 1 wherein the carrier is a milk or milk product.

4. The method of claim 4 wherein the milk product is chocolate flavored.

5. The method of claim 1 wherein the product is a powder containing P. acidilactici NRRL B-50517 and the carrier is a dried fruit or vegetable powder.

6. The method of claim 1 wherein the P. acidilactici NRRL B-50517 is the carrier is a pureed fruit.

7. The method of claim 5 wherein the carrier is a dried fruit powder.
